(19) United States
(12) Patent Application Publication
(10) Pub. No.: US 2022/0315890 A1
(43) Pub. Date: Oct. 6, 2022

(54) NERVE CELL AND APPLICATION THEREOF

(71) Applicants: FUJIFILM Corporation, Tokyo (JP); KEIO UNIVERSITY, Tokyo (JP)

(72) Inventors: Hirokazu TANABE, Ashigarakami-gun (JP); Setsu ENDOH, Ashigarakami-gun (JP); Hideyuki OKANO, Tokyo (JP); Sumihiro MAEDA, Tokyo (JP)

(73) Assignees: FUJIFILM Corporation, Tokyo (JP); KEIO UNIVERSITY, Tokyo (JP)

(21) Appl. No.: 17/709,565

(22) Filed: Mar. 31, 2022

(30) Foreign Application Priority Data
Mar. 31, 2021 (JP) 2021-061794

(51) Int. Cl. C12N 5/079

(52) U.S. Cl. CPC C12N 5/0618; C12N 2506/45

(57) ABSTRACT

An object of the present invention is to provide a nerve cell in which tau aggregation and cell death are caused; a screening kit and a screening method, in which the nerve cell is used; a drug candidate substance obtained by the screening method; a human pluripotent stem cell for producing the nerve cell; and a method of producing the nerve cell.

There is provided a nerve cell having an introduced exogenous wild-type tau gene.

### CROSS-REFERENCE TO RELATED APPLICATIONS

This application claims priority under 35 U.S.C 119 to Japanese Patent Application No. 2021-061794 filed on Mar. 31, 2021. Each of the above applications is hereby expressly incorporated by reference, in its entirety, into the present application.

### BACKGROUND OF THE INVENTION

### 1. Field of the Invention

The present invention relates to a nerve cell that exhibits tau aggregation and cell death by an expression of a tau protein. In addition, the present invention relates to a screening kit and a screening method, using the nerve cell. In addition, the present invention relates to a drug candidate substance obtained by the screening method. Further, the present invention relates to a human pluripotent stem cell and a method of producing a nerve cell.

### 2. Description of the Related Art

Since the development mechanism of a neurodegenerative disease such as Alzheimer's disease is diverse, the research and development of therapeutic drugs based on various mechanisms are expected. Tau is conceived to be a major factor involved in the development of the neurodegenerative disease and increases in the brain of a patient with a neurodegenerative disease. In addition, it has been reported that there is a correlation between the amount of tau accumulated and the decrease in cognitive function, and thus tau is expected as a therapeutic target for neurodegenerative diseases.

As one of mechanisms by which tau causes neurodegenerative diseases, it has been reported that aggregated tau causes nerve cell death. The tau aggregation progresses to neurofibrillary tangle through the tau dissociation from microtubules, the formation of tau oligomers by polymerization, and then the formation of the fibrous tau. However, it remains still unclear how the tau aggregate causes nerve cell death in nerve cells and how the nerve cell death can be controlled. As a result, a nerve cell model in which tau aggregation and nerve cell death occur is expected to be established, as well as the research and development of a novel drug utilizing such a model.

Comput Struct Biotechnol J. 2014 Oct. 2; 12 (20-21): 7-13 describes a method of introducing a tau expression vector from the outside into a non-nerve cell that does not express tau to artificially express tau and quantifying the tau by various methods. J Biomol Screen. 2016 September; 21 (8): 804-15 describes that tau aggregation and cell death are induced by adding tau aggregation plates (which are obtained by aggregating tau fragments) in addition to the overexpression of tau by using a Tet-on adeno-associated virus (AAV) vector in a human iPS cell-derived neuronal precursor cell (NPC). In the method described in J Biomol Screen. 2016 September; 21 (8): 804-15, a long evaluation period of 30 to 40 days is required so that differentiation is induced while the tau gene is introduced into the NPC. Further, a large amount of AAV, that is, a multiplicity of infection (MOI) of 100 is used.

WO2016/076435A describes that a patient-derived iPS cell-derived nerve cell having a mutant tau gene is used to reproduce tau aggregation and nerve cell death; however, it does not describe Examples regarding the introduction of an exogenous tau gene. By the way, it has also been reported that tau aggregation and cell death are caused by mutant tau due to the reason that tau aggregation and cell death have been observed in nerve cells carrying a mutant tau gene. JP2008-220302A describes that an exogenous mutant γPKC-GFP is introduced into CHO cells, the expression thereof is controlled by tet-off, and the aggregation suppression by trehalose is evaluated; however, it does not describe Examples regarding the introduction of an exogenous tau gene. WO2009/101942A describes that an H4-tau cell, which is a neuroblast into which tau is introduced, and a murine nerve cell are used, and tau is phosphorylated by overexpression of PRKX, which causes aggregation and cell death. The invention of WO2009/101942A is for evaluating the influence of PRKX, and thus tau does not aggregate in a case where PRKX is not present.

### SUMMARY OF THE INVENTION

As methods of evaluating the aggregation or the toxicity of intracellular tau in the related art, a method of overexpressing a mutant tau, a method of treating an agglutination promoting agent in addition to overexpression of a mutant tau (Comput Struct Biotechnol J. 2014 Oct. 2; 12 (20-21): 7-13 and J Biomol Screen. 2016 September; 21 (8): 804-15), and further a method of using a human pluripotent stem cell-derived nerve cell, where the human pluripotent stem cell has been established from a patient cell, (WO2016/076435A) are known. The method of overexpressing a mutant tau has the disadvantage that the pathophysiology caused by the aggregation of the wild-type tau cannot be reproduced. In addition, the method of using a tau aggregation promoting agent in combination has defects in that it is difficult to evaluate the phenotype of tau alone since the tau aggregation promoting agent itself is toxic. In addition, in the method of using a human pluripotent stem cell-derived nerve cell, where the human pluripotent stem cell has been established from a patient cell, a long-term culture is generally required for the phenotypic expression, and thus it has been difficult to carry out drug screening.

An object to be achieved by the present invention is to provide a nerve cell in which tau aggregation and cell death are caused. In addition, another object to be achieved by the present invention is to provide a screening kit and a screening method, using the nerve cell. In addition, another object to be achieved by the present invention is to provide a drug candidate substance obtained by the screening method. Further, another object to be achieved by the present invention is to provide a human pluripotent stem cell for producing the nerve cell and a method of producing the nerve cell.

As a result of diligent studies to achieve the above objects, the inventors of the present inventions have succeeded in causing tau aggregation and cell death by introducing an exogenous wild-type tau gene into a nerve cell. The present invention has been completed based on the above findings.

That is, according to the present invention, the following inventions are provided.

<1> A nerve cell comprising an introduced exogenous wild-type tau gene.

<2> The nerve cell according to <1>, in which the nerve cell is derived from a human pluripotent stem cell.

<3> The nerve cell according to <2>, in which the human pluripotent stem cell is a human pluripotent stem cell derived from a healthy subject.

<4> The nerve cell according to <2> or <3>, in which the exogenous wild-type tau gene is expressed after differentiation of the human pluripotent stem cells into the nerve cell.

<5> The nerve cell according to any one of <1> to <4>, in which an expression of the exogenous wild-type tau gene is controlled in a condition-specific manner.

<6> The nerve cell according to any one of <1> to <5>, in which tau aggregates in a case where the exogenous wild-type tau gene is overexpressed.

<7> The nerve cell according to any one of <1> to <6>, in which aggregation of tau occurs due solely to a gene expression.

<8> A screening kit for a drug candidate substance having an action on a change due to overexpression of tau, the screening kit comprising the nerve cell according to any one of <1> to <7>.

<9> A screening method for a drug candidate substance having an action of suppressing tau aggregation or cell death, the screening method comprising using the nerve cell according to any one of <1> to <7>.

<10> A drug candidate substance obtained by the screening method according to <9>.

<11> A human pluripotent stem cell comprising an introduced exogenous wild-type tau gene.

<12> A method of producing a nerve cell, comprising:

introducing an exogenous wild-type tau gene into a human pluripotent stem cell; and

differentiating the human pluripotent stem cell into a nerve cell.

<13> The method according to <12>, in which steps of an expression of the exogenous wild-type tau gene and a gene expression for nerve cell differentiation are provided by a TET system.

According to the nerve cell according to the aspect of the present invention, it is possible to cause tau aggregation and cell death only by the expression of the wild-type tau.

### BRIEF DESCRIPTION OF THE DRAWINGS

FIG. 1 is a map showing a CSIV-miR-9/9*-124-mRFP1-TRE-EF-BsdT vector.

FIG. 2 a map showing a CSII-TRE-hTau (1N4R)-IRES-Zeo vector.

FIG. 3 is a schematic view of a preparation method for a nerve cell and overexpression of tau by a lentivirus.

FIG. 4 is an image showing the detection of overexpressed tau by sodium dodecyl sulfate-polyacrylamide gel electrophoresis (SDS-PAGE).

FIG. 5 is images showing neurite degeneration due to overexpression of tau in human induced pluripotent stem cell (hiPSC)-derived nerve cells.

FIG. 6 is a graph showing the detection of nerve cell death due to overexpression of tau.

FIG. 7 is images and a graph, showing the detection of aggregated tau in neurites due to overexpression of tau.

FIG. 8 is graphs showing the drug efficacy of tau polymerization inhibitors and microtubule stabilizers.

### DESCRIPTION OF THE PREFERRED EMBODIMENTS

Hereinafter, the content of the present invention will be described in detail.

In the present specification, “NP_”, “NM_”, and “NG_” together with numbers following them respectively represent an amino acid sequence (NP_˜), a nucleotide sequence (NM_˜) of a transcript, and an ID of a genomic DNA sequence (NG_˜), which are registered as the reference sequences in the National Center for Biotechnology Information (NCBI) database.

Nerve Cell

The nerve cell according to the embodiment of the present invention is a nerve cell having an introduced exogenous wild-type tau gene.

Preferably, the nerve cell according to the embodiment of the present invention is a cell derived from a pluripotent stem cell.

The “pluripotent stem cell” refers to a cell having the ability (the differentiation pluripotency) to differentiate into all cells that constitute a living body and the ability (the self-replication ability) to generate a daughter cell having the same differentiation potency as the mother cell through cell division. The differentiation pluripotency can be evaluated by transplanting an evaluation target cell into a nude mouse and testing for the presence or absence of formation of teratoma that includes cells of the respective three germ layers (ectoderm, mesoderm, and endoderm).

Examples of the pluripotent stem cell include an embryonic stem cell (an ES cell), an embryonic germ cell (an EG cell), and an induced pluripotent stem cell (an iPS cell); however, examples thereof are not limited thereto as long as a cell has both differentiation pluripotency and self-replication ability. An ES cell or an iPS cell is preferably used. An iPS cell is more preferably used. The pluripotent stem cell is preferably a mammalian (for example, primates such as a human or a chimpanzee, rodents such as a mouse or a rat) cell. The pluripotent stem cell is preferably a human pluripotent stem cell and more preferably a human pluripotent stem cell derived from a healthy subject. In the most preferred aspect of the present invention, a human iPS cell is used as the pluripotent stem cell.

The ES cell can be established, for example, by culturing an early embryo before implantation, an inner cell mass constituting the above early embryo, or a single blastomere (Manipulating the Mouse Embryo, A Laboratory Manual, Second Edition, Cold Spring Harbor Laboratory Press (1994); Thomason, J. A. et al., Science, 282, 1145-1147 (1998)). As the early embryo, an early embryo prepared by nuclear transfer of a somatic cell nucleus may be used (Wilmut et al. (Nature, 385, 810 (1997)), Cibelli et al. (Science, 280, 1256) (1998)), Akira Iriya et al. (Protein, nucleic acid, and enzyme, 44, 892 (1999)), Baguisi et al. (Nature Biotechnology, 17, 456 (1999)), Wakayama et al. (Nature, 394, 369 (1998)); Nature Genetics, 22, 127 (1999); Proc. Natl. Acad. Sci. USA, 96, 14984 (1999)), Rideout III et al. (Nature Genetics, 24, 109 (2000), Tachibana et al. (Human Embryonic Stem Cells Delivered by Somatic Cell Nuclear Transfer, Cell (2013) in press). As the early embryo, a parthenogenetic embryo may be used (Kim et al. (Science, 315, 482-486 (2007)), Nakajima et al. (Stem Cells, 25, 983-985 (2007)), Kim. et al. (Cell Stem Cell, 1,346-352 (2007)), Revazova et al. (Cloning Stem Cells, 9, 432-449 (2007)), Revazova et al. (Cloning Stem Cells, 10, 11-24 (2008)). In addition to the above-described papers, regarding the preparation of an ES cell, the following can be referenced, Strelchenko N. et al. Reprod Biomed Online. 9: 623-629, 2004; Klimanskaya I., et al. Nature 444: 481-485, 2006; Chung Y., et al. Cell Stem Cell 2: 113-117, 2008; Zhang X., et al. Stem Cells 24: 2669-2676, 2006; Wassarman, P. M. et al. Methods in Energy, Vol. 365, 2003, and the like. It is noted that a fused ES cell obtained by cell fusion of an ES cell with a somatic cell is also included in the embryonic stem cell that is used in the method according to the embodiment of the present invention.

Some ES cells are available from conservation institutions or are commercially available. For example, human ES cells are available from the Institute for Frontier Medical Sciences, Kyoto University (for example, KhES-1, KhES-2, and KhES-3), WiCell Research Institute, ESI BIO, and the like.

The EG cell can be established by, for example, culturing a primordial germ cell in the presence of a leukemia inhibitory factor (LIF), a basic fibroblast growth factor (bFGF), and a stem cell factor (SCF) (Matsui et al., Cell, 70, 841-847 (1992), Shamblott et al., Proc. Natl. Acad. Sci. USA, 95 (23), 13726-13731 (1998), Turnpenny et al., Stem Cells, 21 (5), 598-609, (2003)).

“Induced pluripotent stem cell (iPS cell)” is a cell having pluripotency (multiple differentiation potency) and proliferation ability, which is prepared by reprogramming a somatic cell by introducing reprogramming factors or the like. The induced pluripotent stem cell exhibits properties similar to the ES cell. The somatic cell that is used for preparing an iPS cell is not particularly limited and may be a differentiated somatic cell or an undifferentiated stem cell. In addition, the origin of the somatic cell is not particularly limited: however, a somatic cell of a mammal (for example, primates such as a human or a chimpanzee, rodents such as a mouse or a rat) cell is preferably used, and a human cell particularly preferably used. The iPS cell can be prepared by various methods reported so far. In addition, it is naturally expected that an iPS cell preparation method to be developed in the future will be applied.

The most basic method of preparing an iPS cell is a method of introducing four transcription factors, Oct3/4, Sox2, Klf4, and c-Myc, into a cell using a virus (Takahashi K, Yamanaka S: Cell 126 (4), 663-676, 2006; Takahashi, K, et al: Cell 131 (5), 861-72, 2007). It has been reported that human iPS cells have been established by introducing four factors, Oct4, Sox2, Lin28, and Nanog (Yu J, et al.: Science 318 (5858), 1917-1920, 2007). It has also been reported that iPS cells are established by introducing three factors excluding c-Myc (Nakagawa M, et al: Nat. Biotechnol. 26 (1), 101-106, 2008), two factors of Oct3/4 and Klf4 (Kim J B, et al: Nature 454 (7204), 646-650, 2008), or only Oct3/4 (Kim J B, et al: Cell 136 (3), 411-419, 2009). In addition, a method of introducing a protein, which is an expression product of a gene, into a cell (Zhou H, Wu S, Joo J Y, et al: Cell Stem Cell 4, 381-384, 2009; Kim D, Kim C H, Moon J I, et al.: Cell Stem Cell 4, 472-476, 2009) has also been reported. On the other hand, it has been also reported that it is possible to improve the preparation efficiency or reduce the factors to be introduced, by using an inhibitor BIX-01294 for a histone methyltransferase G9a, a histone deacetylase inhibitor valproic acid (VPA), Bay K8644, or the like (Huangfu D, et al: Nat. Biotechnol. 26 (7), 795-797, 2008; Huangfu D, et al: Nat. Biotechnol. 26 (11), 1269-1275, 2008; Silva J, et al: PLoS. Biol. 6 (10), e253, 2008). In addition, gene introducing methods have been studied as well, and techniques using, in addition to retroviruses, the following substances have been developed; lentiviruses (Yu J, et al: Science 318 (5858), 1917-1920, 2007), adenoviruses (Stadtfeld M, et al: Science 322 (5903), 945-949, 2008), plasmids (Okita K, et al: Science 322 (5903), 949-953, 2008), transposon vectors (Woltjen K, Michael I P, Mohseni P, et al: Nature 458, 766-770, 2009; Kaji K, Norrby K, Pac a A, et al: Nature 458, 771-775, 2009; Yusa K, Rad R, Takeda J, et al: Nat Methods 6,363-369, 2009), or episomal vectors (Yu J, Hu K, Smuga-Otto K, Tian S, et al: Science 324, 797-801, 2009).

Cells transformed to iPS cells, that is, cells that have undergone initialization (reprogramming) can be selected using, as an indicator, the expression of pluripotent stem cell markers (undifferentiated markers) such as Fbxo15, Nanog, Oct3/4, Fgf-4, Esg-1, and Cript, or the like. The selected cells are collected as the iPS cell.

iPS cells can be provided from FUJIFILM Cellular Dynamics, Inc.; National University Corporation, Kyoto University; or Independent Administrative Institution, Institute of Physical and Chemical Research, BioResource Research Center.

Pluripotent stem cells can be cultured using a medium suitable for culturing pluripotent stem cells. In a case where iPS cells are used as pluripotent stem cells, for example, StemFit (registered trade name) AKO2N (Ajinomoto Co., Inc.), mTeSR (registered trade name) 1 (Stemcell Technologies), StemFlex (registered trade name), and the like can be used. The culture may be carried out on a plate (for example, a 6-well plate or the like) or in a flask; however, it is preferably carried out on a plate. The culture period is not particularly limited, and it is possible to carry out culture, for example, for 1 to 7 days.

The nerve cell according to the embodiment of the present invention has an exogenous wild-type tau gene introduced from the outside.

Tau (microtubule-associated protein tau, also called MAPT) is a protein encoded by an MAPT gene (official full name: microtubule-associated protein Tau, official symbol: MAPT, NG_007398.1) located on chromosome 17 (17q21.1) in humans, and six isoforms produced by selective splicing have been identified.

The number (0 to 2) of characteristic amino acid sequences (N) of 29 amino acids on the N-terminal side and the number (3 or 4) of repetitive sequences (R) involved in microtubule binding on the C-terminal side are different between the isoforms, and thus depending on the number of these sequences, each of the isoforms is classified into a 0N3R type (352 amino acids, NP_058525.1, NM_016841.4), a 1N3R type (381 amino acids, NP_001190180.1, NM_001203251.1), a 2N3R type (410 amino acids, NP_001190181.1, NM_001203252.1), a 0N4R type (383 amino acids, NP_058518.1, NM_016834.4), a 1N4R type (412 amino acids, NP_001116539.1, NM_001123067.3), and a 2N4R type (441 amino acids, NP_005901.2, NM_005910.5) (regarding each of the human isoforms, the number of amino acid residues, the ID of the reference amino acid sequence, and the ID of the reference nucleotide sequence of the transcript are shown in parentheses as an example).

The tau gene in the present invention may be a tau gene of a mammal (for example, rodents such as a mouse and a rat, or primates such as a marmoset) other than the human. There are the following six types of tau protein isoforms that can be generated by alternative splicing in the brain of the mammalian living body: the 0N3R type, the 1N3R type, the 2N3R type, the 0N4R type, 1N4R type, and the 2N4R type, which are described above.

Only 3R type tau is expressed in the human brain in the fetal period. However, all of the above 6 types are expressed in the human adult brain, and the expression ratio (=4 repeat tau/3 repeat tau) of the 4R type (4 repeat tau) to the 3R type (3 repeat tau) in normal humans is about 1.

Regarding mice, only 3R-type tau is expressed up to the newborn mouse stage; however, only 4R-type tau is expressed after the weaning period.

Regarding rats and marmosets as well, only 3R-type tau is expressed up to the newborn rat or marmoset stage; however, only 4R-type tau is expressed after the weaning period.

In the present invention, unless otherwise specified, the tau may be any one of the 3 repeat tau or the 4 repeat tau.

Production of Nerve Cell

The nerve cell according to the embodiment of the present invention can be produced by differentiating a human pluripotent stem cell having an introduced exogenous wild-type tau gene into a nerve cell. The human pluripotent stem cell having an introduced exogenous wild-type tau gene can be produced by introducing the exogenous wild-type tau gene into a human pluripotent stem cell. That is, according to the present invention, there is provided a method of producing a nerve cell, which includes introducing an exogenous wild-type tau gene into a human pluripotent stem cell and differentiating the human pluripotent stem cell into a nerve cell.

The method of inducing the differentiation of a pluripotent stem cell into a nerve cell from is not particularly limited; however, it includes a method of preparing a neural stem cell from a pluripotent stem cell by using a treatment with a low-molecular-weight compound and then inducing the neural stem cell to a nerve cell, and a method of carrying out direct induction to a nerve cell by a gene expression or the like. In the present invention, pluripotent stem cells are induced to be differentiated into nerve cells, and thus nerve cells not including glial cells can be produced. The nerve cells according to the embodiment of the present invention are preferably a cell population that does not include glial cells. Further, the nerve cells according to the embodiment of the present invention are preferably cells which are not cultured in three dimensions (in a three-dimensional manner) and more preferably cells cultured in a single layer.

Examples of the method of inducing the differentiation of a pluripotent stem cell into a nerve cell include:

(1) a method of carrying out culture in a serum-free medium to form an embryoid body (a cell mass containing neuronal precursor cells) and differentiating the embryoid body (an SFEB method: Watanabe K., et al, Nat. Neurosci., 8: 288-296, 2005; SFEBq method: Wataya T., et al, Proc. Natl. Acad. Sci. USA., 105: 11796-11801, 2008);

(2) a method of carrying out culture and differentiation on the stroma cell (an SDIA method: Kawasaki H., et al, Neuron, 28: 31-40, 2000);

(3) a method of carrying out culture and differentiation on Matrigel to which a drug has been added (Chambers S. M., et al, Nat. Biotechnol., 27: 275-280, 2009);

(4) a method carrying out culture and differentiation in a medium containing a low-molecular-weight compound as a substitute for a cytokine (U.S. Pat. No. 5,843,780A);

(5) a method of introducing a neural inducing factor (neurogenin 2 (Ngn2) or the like) into a pluripotent stem cell and expressing the nerve-inducing factor (WO2014/148646A; and Zhang Y., et al, Neuron, 78: 785-98, 2013) to carry out the differentiation of the pluripotent stem cell;

(6) a method of introducing miR-9/9*-124 into a pluripotent stem cell and expressing the miR-9/9*-124 to carry out the differentiation of the pluripotent stem cell; and

a combination of these methods.

Among them, (5) the method of introducing neurogenin 2 into a pluripotent stem cell and expressing the neurogenin 2 is preferable since mature nerve cells can be obtained in a short period of time and with high efficiency. Further, (6) the method of introducing miR-9/9*-124 into a pluripotent stem cell and expressing the miR-9/9*-124 to carry out the differentiation of the pluripotent stem cell is also preferable. The method of inducing the differentiation of a pluripotent stem cell into a nerve cell is preferably a method of carrying out direct induction to a nerve cell by expressing Ngn2 alone or Ngn2 and miR-9/9*-124. It is most preferably a method of carrying out induction to a nerve cell by expressing Ngn2 alone or Ngn2 and miR-9/9*-124 with a TET-on promoter.

The neurogenin 2 protein is a transcription factor known to promote differentiation into nerve cells during development, and the amino acid sequence thereof is exemplified by NP_076924 in humans and NP_033848 in mice. The neurogenin 2 gene (official full name: neurogenin 2, official symbol: NEUROG2, also called the Ngn2 gene) is a DNA encoding the neurogenin 2 protein, and examples thereof include NM_009718 (mouse) or NM_024019 (human) registered as the reference sequence and a DNA having a nucleotide sequence of a transcript variant thereof. Further, it may be a DNA having complementarity to the extent by which the DNA can be hybridized to the reference sequence or the nucleic acid having a sequence of the transcript variant under stringent conditions.

The stringent conditions can be determined based on the melting temperature (Tm) of the nucleic acid to which a complex or a probe binds. Examples of the washing conditions after hybridization typically include conditions of about “1× saline sodium citrate buffer (SSC), 0.1% SDS, 37° C.”. It is preferable that a complementary strand maintains a state of being hybridized with a target positive strand even in a case of being washed under such conditions. Further, examples of the washing conditions include conditions under which a positive strand maintains a state of being hybridized with a complementary strand even in a case of being washed under washing conditions of about “0.5×SSC, 0.1% SDS, 42° C.” as the more severe hybridization conditions and washing conditions of about “0.1×SSC, 0.1% SDS, 65° C.” as the still more severe hybridization conditions. Specific examples of such a complementary strand include a strand consisting of a base sequence having a completely complementary relationship with a base sequence of a target positive strand, and a strand consisting of a base sequence having at least 90%, preferably 95% or more, more preferably 97% or more, still more preferably 98% or more, and particularly preferably 99% or more identity with the above complementary strand.

The expression of neurogenin 2 in the pluripotent stem cell can be carried out by introducing a nucleic acid (DNA or RNA) encoding neurogenin 2 or a neurogenin 2 (protein) into the pluripotent stem cell.

The expression of miR-9/9*-124 in the pluripotent stem cell can be carried out by introducing a nucleic acid (DNA or RNA) encoding miR-9/9*-124 into the pluripotent stem cell.

In the present invention, in a case of introducing a nucleic acid encoding neurogenin 2 and a nucleic acid encoding miR-9/9*-124 into a cell, it is possible to introduce, for example, a vector such as a virus, a plasmid, or an artificial chromosome into a pluripotent stem cell by using a method such as lipofection, a method using a liposome, microinjection, or the like. Examples of the virus vector include a retrovirus vector, a lentivirus vector, an adenovirus vector, an adeno-associated virus vector, and a Sendai virus vector. In addition, the artificial chromosome vector includes, for example, a human artificial chromosome (HAC), a yeast artificial chromosome (YAC), a bacterial artificial chromosome (BAC, PAC), and the like. As the plasmid, a plasmid for mammals can be used. The vector can contain regulatory sequences for expressing the neurogenin 2 protein or the miR-9/9*-124 (a promoter, an enhancer, a ribosome binding sequence, a terminator, a polyadenylation site, and the like) and, as desired, it may further contain a drug resistance gene (for example, a kanamycin resistance gene, an ampicillin resistance gene, a puromycin resistance gene, or the like), a selection marker sequence such as a thymidine kinase gene or a diphtheria toxin gene, and a reporter gene sequence such as β-glucuronidase (GUS), FLAG, or the like. In particular, it is preferable that the nucleotide sequence encoding the amino acid sequence of the protein is functionally conjugated to an inducible promoter sequence so that the expression of the neurogenin 2 protein or miR-9/9*-124 can be rapidly induced at the desired time.

Examples of the inducible promoter include drug-responsive promoters, and suitable examples thereof include a tetracycline-responsive promoter (a CMV minimal promoter having a tetracycline-responsive sequence (TRE) in which seven tetO sequences are consecutively included). For example, a Tet-On/Off Advanced expression induction system is exemplified; however, a Tet-On system is preferable since it is desirable that the gene of interest can be expressed in the presence of tetracycline. That is, it is a system in which a reverse tetR (rtetR) and a fusion protein (rtTA) fused with VP16AD are simultaneously expressed. The Tet-On system can be available from Clontech and used. In addition, it is also possible to suitably use a cumate-responsive promoter (Q-mate system, Krackeler Scientific, Inc., National Research Council (NRC), or the like), an estrogen-responsive promoter (WO2006/129735A, and a GenoStat inducible expression system, Upstate Cell Signaling Solutions), an RSL1-responsive promoter (RheoSwitch mammal inducible expression system, New England Biolabs), or the like. Among the above, a tetracycline-responsive promoter or a cumate-responsive promoter is particularly preferable, and a tetracycline-responsive promoter is most preferable, due to the high specificity and the low toxicity of an expression-induced substance.

In a case of using a cumate-responsive promoter, it is suitable that a mode in which a CymR repressor is expressed in a pluripotent stem cell is provided together.

Further, the regulatory sequence and the regulatory factor of the above promoter (the rtTA and/or the CymR repressor, or the like) may be supplied by a vector into which the neurogenin 2 gene or miR-9/9*-124 has been introduced.

In a case of using a tetracycline-responsive promoter, it is possible to maintain the expression of neurogenin 2 or miR-9/9*-124 by continuously adding tetracycline or a derivative thereof, doxycycline (hereinafter, abbreviated as DOX in the present application), in the medium for a desired period of time. In addition, in a case of using a cumate-responsive promoter, it is possible to maintain the expression of neurogenin 2 or miR-9/9*-124 by continuously adding a cumate in the medium for a desired period of time.

As the promoter for expressing the neurogenin 2 protein or the miR-9/9*-124, a constitutive expression promoter (a cytomegalovirus (CMV)-derived promoter, an EF1 promoter, a ubiquitin C (UbC) promoter, a murine stem cell virus (MSCV)-derived promoter, or the like), a nerve-specific promoter (a Syn promoter or the like), or the like may be used.

Even in a case of using a drug-responsive promoter, it is possible to stop the expression of neurogenin 2 or miR-9/9*-124 by removing the corresponding drug from the medium (for example, by replacing the medium with a drug-free medium).

In the present invention, in a case where a nucleic acid encoding neurogenin 2, or miR-9/9*-124 is introduced in the form of RNA, it may be introduced into pluripotent stem cells by a method such as electroporation, lipofection, or microinjection. In order to maintain the expression of neurogenin 2 or miR-9/9*-124 in cells, the introduction may be carried out a plurality of times, for example, twice, three times, four times, or five times.

In the present invention, in a case where neurogenin 2 is introduced in the form of a protein, it may be introduced into pluripotent stem cells, for example, by a method such as lipofection, a method of using fusion with a cell membrane-permeable peptide (for example, an HIV-derived TAT or a polyarginine), microinjection, or the like. In order to maintain the expression of neurogenin 2 in cells, the introduction may be carried out a plurality of times, for example, twice, three times, four times, or five times.

In the present invention, the period in which neurogenin 2 or miR-9/9*-124 is expressed in pluripotent stem cells for nerve cell induction is not particularly limited; however, in a case of human pluripotent stem cells, it is 2 days or more, preferably 3 days or more, and still more preferably 4 days or more.

After the expression induction in the method of carrying out differentiation by introducing neurogenin 2 or miR-9/9*-124, the pluripotent stem cells are preferably cultured in a medium (which is referred to as a medium for nerve differentiation) suitable for inducing differentiation into nerve cells. As such a medium, only the basal medium or a basal medium to which a neurotrophic factor is added can be used. The neurotrophic factor in the present invention is a ligand for a membrane receptor that plays an important role in the survival and the maintenance of function of nerve cells, and examples thereof include nerve growth factor (NGF), brain-derived neurotropic factor (BDNF), neurotrophin 3 (NT-3), neurotrophin 4/5 (NT-4/5), neurotrophin 6 (NT-6), basic FGF, acidic FGF, FGF-5, epidermal growth factor (EGF), hepatocyte growth factor (HGF), insulin, insulin-like growth factor 1 (IGF1), insulin-like growth factor 2 (IGF-2), glia cell line-derived neurotropic factor (GDNF), TGF-b2, TGF-b3, interleukin 6 (IL-6), ciliary neurotropic factor (CNTF), and LIF. Among these, the preferred neurotrophic factor is GDNF, BDNF, and/or NT-3.

Examples of the basal medium include a Glasgow's Minimal Essential Medium (GMEM) medium, an IMDM medium, a Medium 199 medium, an Eagle's Minimum Essential Medium (EMEM) medium, an aMEM medium, a Dulbecco's modified Eagle's Medium (DMEM) medium, a Ham's F12 (F12) medium, a Dulbecco's Modified Eagle Medium: Nutrient Mixture F-12 (DMEM/F-12) medium, an RPMI 1640 medium, a Fischer's medium, a Neurobasal Medium medium (Thermo Fisher Scientific, Inc.), a Neurobasal Plus Medium medium (Thermo Fisher Scientific, Inc.), and BrainPhys (Stemcell Technologies), as well as a mixed medium thereof.

The basal medium may contain serum or may be serum-free. As necessary, the medium may contain one or more serum substitutes, for example, Knockout Serum Replacement (KSR) (a serum substitute for FBS during ES cell culture), an N2 supplement (Thermo Fisher Scientific, Inc.), a B27 supplement (Thermo Fisher Scientific, Inc.), a B27 Plus supplement (Thermo Fisher Scientific, Inc.), a Culture One supplement (Thermo Fisher Scientific, Inc.), albumin, transferrin, apotransferrin, fatty acid, insulin, a collagen precursor, trace elements, 2-mercaptoethanol, and 3′-thiol glycerol, and may also contain one or more substances such as a lipid, an amino acid, L-glutamine, Glutamax (Thermo Fisher Scientific, Inc.), a non-essential amino acid, a vitamin, a growth factor, a low-molecular-weight compound, an antibiotic, an antioxidant, pyruvic acid, a buffer, an inorganic salt, selenic acid, progesterone, and putrescine.

In the present invention, as the medium for nerve differentiation, it is particularly preferable to use a medium obtained by adding a B27 Plus supplement, a Culture One supplement, Glutamax, dbcAMP, L-ascorbic acid, Y27632, and N—[N-(3,5-difluorophenacetyl-L-alanyl)]-(S)-phenylglycine t-butyl ester (DAPT) to Neurobasal plus Medium.

The culture temperature at the time of inducing the differentiation into the nerve cell, the culture temperature is not particularly limited; however, it is about 30° C. to 40° C. and preferably about 37° C. The culture is carried out in an atmosphere of the CO₂-containing air, where the CO₂ concentration is preferably about 2% to 5%.

The nerve cell in the present invention preferably a cell that expresses at least one of marker genes specific to the nerve cell, consisting of β-III tubulin, NeuN, neural cell adhesion molecule (N-CAM), and microtubule-associated protein 2 (MAP2), and has β-III tubulin-positive protrusion (hereinafter, referred to as a neurite).

The more preferred nerve cell in the present invention is a morphologically mature nerve cell, and the still more preferred one is a glutamatergic nerve cell. Here, the morphologically mature nerve cell is a nerve cell in which the cell body is hypertrophic and the neurite is sufficiently extended (as a guide, the neurite length is about 5 times or more of the diameter of the cell body).

The nerve cell according to the embodiment of the present invention can be produced by introducing an exogenous wild-type tau gene into a nerve cell obtained by inducing differentiation of a pluripotent stem cell as described above. Alternatively, the nerve cell according to the embodiment of the present invention may be produced by introducing an exogenous wild-type tau gene into a pluripotent stem cell before inducing differentiation into a nerve cell, and then inducing differentiation of this pluripotent stem cell into a nerve cell.

Examples of the method of introducing an exogenous wild-type tau gene include a method using a viral vector, lipofection, and electroporation. A viral vector is preferable, a lentivirus, an adeno-associated virus (AAV), a Sendai virus, a retrovirus, or the like is more preferable, and a lentivirus is most preferable.

A promoter is preferably used for overexpression of an exogenous wild-type tau. Preferably, a constitutive expression promoter (a cytomegalovirus (CMV)-derived promoter, an EF1 promoter, a ubiquitin C (UbC) promoter, a murine stem cell virus (MSCV)-derived promoter, or the like), a nerve-specific promoter (a Syn promoter or the like), or a drug-inducible promoter (for example, a TET-on promoter, a TET-off promoter, an isopropyl-β-thiogalactopyranoside (IPTG) promoter, or the like) can be used. More preferably, a TET-on promoter can be used.

It is preferable that in the nerve cell according to the embodiment of the present invention, the exogenous wild-type tau gene is expressed after the differentiation of the human pluripotent stem cells into the nerve cell. In addition, it is preferable that the expression of the exogenous wild-type tau gene is controlled in a condition-specific manner.

In the present invention, steps of the expression of the exogenous wild-type tau gene and the gene expression for nerve cell differentiation are particularly preferably provided by a TET system (a TET-on system or a TET-off system). The TET system is a system capable of reversibly regulating the expression of a target gene in cells by administration of doxycycline, which is a tetracycline derivative.

In the nerve cell according to the embodiment of the present invention, tau aggregates in a case where the exogenous wild-type tau gene is overexpressed. The tau aggregation is preferably due solely to the expression of the exogenous wild-type tau gene.

In one example of the preferred embodiment of the present invention, a human nerve cell can be prepared from a human pluripotent stem cell. Preferably, the nerve cell is prepared in a manner of the TET-on inducible expression of Ngn2 and miR-9/9*-124. Preferably, prepared nerve cells are frozen and stocked and then used by being thawed at the time of evaluation. The prepared nerve cells are preferably plated in a coated cell culture plate; however, the nerve cells are more preferably plated in a single layer. After replating the nerve cells, the tau gene can be exogenously introduced to express tau. Particularly preferably, a lentivirus vector can be used to express tau in a TET-on inducible manner by treatment with doxycycline.

Screening Method

The present invention relates to a screening kit for a drug candidate substance having an action on a change due to overexpression of tau, where the screening kit includes the nerve cell described above, and a screening method for a drug candidate substance having an action of suppressing tau aggregation or cell death, where the screening method includes using the nerve cell described above. Further, the present invention relates to a drug candidate substance obtained by the screening method described above.

In the nerve cell according to the embodiment of the present invention, tau aggregation and nerve cell death can be induced by overexpression of tau, and thus the nerve cell according to the embodiment of the present invention can be used in the screening of drug candidate substances for central nervous system diseases and the analysis of pathophysiological mechanisms. That is, in the nerve cell according to the embodiment of the present invention, the tau aggregation and the nerve cell death associated with tau aggregation can be manifested only by the expression of wild-type tau. In addition, drug candidate substances can be screened using these phenotypes as indicators. In the present invention, it is possible to obtain effects of not only nerve cell death but also fragmentation and/or disappearance of neurites, an increase in aggregated tau, and an increase in phosphorylated tau. In addition, it is possible to evaluate the response of drugs to these phenotypes.

In the present invention, by an evaluation on live cells or dead cells, western blotting, enzyme-linked Immunosorbent assay (ELISA), or a biochemical analysis by staining after overexpressing tau in the nerve cell according to the embodiment of the present invention, it is possible to evaluate tau aggregation, tau phosphorylation, tau amount, tau localization, and the like, and furthermore, it is possible to evaluate neurites, various nerve-related proteins, disease markers, and the like.

In one aspect of the present invention, tau can be aggregated, and cell death can be induced without carrying out additional tau aggregation promoting operation other than the overexpression of wild-type tau. Examples of the tau aggregation promoting operation include the formation of an aggregate of tau fragments (K18 or the like), a treatment with a dephosphorylation inhibitor (okadaic acid or the like), and a treatment with a protein aggregation promoting agent (Congo Red or the like).

In the present invention, the overexpression means that a large amount of target mRNA or target protein is expressed as compared with a case where no operation is carried out. The overexpression preferably means that an amount equal to or more than the endogenous protein is expressed.

The present invention provides a method of screening a prophylactic or therapeutic drug for tauopathy, including, for example:

(1) a step of bringing the nerve cell according to the embodiment of the present invention into contact with a test substance;

(2) a step of overexpressing tau in the above nerve cell;

(3) a step of measuring tau aggregation or nerve cell death associated with tau aggregation in the above nerve cell; and

(4) a step of selecting the test substance as a prophylactic drug or therapeutic drug for tauopathy in a case where tau aggregation or nerve cell death associated with tau aggregation, which has been measured in the step (3) in a case where the nerve cell has been brought into contact with the test substance in the step (1), is decreased to a level lower than that of tau aggregation or nerve cell death associated with tau aggregation in a case where the nerve cell has not been brought into contact with the test substance in the step (1).

The step (1) is a step of plating nerve cells in a culture plate to which a culture solution containing a test substance has been added, or plating nerve cells in a culture plate and then adding a test substance thereto. The addition of the test substance may be carried out before or after the treatment of overexpressing an exogenous tau. Preferably, the test substance is added before tau is overexpressed.

In the step (2), the nerve cells are plated after subjecting the culture plate to which a culture solution has been added, to the treatment of overexpressing tau (for example, the addition of the lentivirus), or the treatment of overexpressing tau is carried out after the nerve cells are plated. Preferably, tau is overexpressed after replating the nerve cells. More preferably, tau is overexpressed 2 days after replating the nerve cells.

In the step (3), nerve cell death can be detected by using a live cell detection reagent such as Cell Titter Glo (Promega Corporation) or Cell counting kit-8 (DOJINDO Co., Ltd.) or using a cell death detection reagent such as propidium iodide or NucGreen Dead (Thermo Fisher Scientific, Inc.). Cell Titer Glo is preferably used. Tau aggregation can be detected by immobilizing nerve cells by a treatment with paraformaldehyde, neutral buffered formalin, ethanol, or the like, and carrying out fluorescent staining using an antibody (a tau antibody, a tau oligomer antibody, a βIII tubulin antibody, or the like). Alternatively, the detection can be carried out by collecting proteins, carrying out electrophoresis of native PAGE, non-reducing SDS-PAGE, SDS-PAGE, or the like, and then using Coomassie Brilliant Blue staining or a tau antibody. Preferably, after the fixation with paraformaldehyde, a tau antibody or a tau oligomer antibody is used for visualization with fluorescent staining. After the visualization, imaging and analysis are carried out using a fluorescence imaging device (In Cell Analyzer (GE Healthcare), IncuCyte (Sartorius AG), or CQ-1 (Yokogawa Electric Corporation)). It is preferable to use In Cell Analyzer. It is more preferable to use In Cell Analyzer 6000.

The analysis is carried out by measuring lengths or fluorescence intensity of neurites which are tau oligomer antibody-positive. An exogenous tau is introduced, neurites in which overexpression is observed are detected, and the protrusion length or the like can be measured by using an antibody (a 4R tau antibody in a case where 1N4R tau is overexpressed in nerve cells in which 4R tau is rarely expressed) that recognizes the overexpressed tau isoform. This indicator can be used as an internal standard for overexpression. Preferably, a value obtained by correcting the tau oligomer antibody-positive protrusion length with the 4R tau antibody-positive protrusion length can be used as an indicator of the tau aggregation.

In the step (4), the case of being decreased to a level lower than that of tau aggregation in a case where the nerve cell has been brought into contact with the test substance in the step (1) is not particularly limited as long as the level is lower; however, a test substance having an action of causing tau aggregation to preferably 80% or less and more preferably 50% or less by bringing the nerve cell into contact with the test substance is selected in a case where tau aggregation in a case where the nerve cell has not been brought into contact with the test substance is set to 100%. In addition, the case of being decreased to a level lower than that of nerve cell death in a case where the nerve cell has been brought into contact with the test substance in the step (1) is not particularly limited as long as the level is lower; however, a test substance having an action of increasing the cell survival rate to preferably 20% or more and more preferably 50% or more by bringing the nerve cell into contact with the test substance is selected as a substance that reduces the nerve cell death, in a case where a value of a cell survival rate in a case where tau has not been overexpressed is set to 100% and a value of a cell survival rate in a case where tau has been overexpressed but the nerve cell has not been brought into contact with the test substance is set to 0%.

The screening method according to the embodiment of the present invention is useful in screening a compound or lead compound that is capable of being a prophylactic or therapeutic drug for tauopathy.

Examples of the tauopathy include Alzheimer's disease (AD), frontotemporal dementia with Parkinsonism linked to chromosome 17 (FTDP-17), frontotemporal dementia (FTD), Pick's disease, progressive supranuclear palsy (PSP), corticobasal degeneration (CBD), argyrophilic grain dementia (argyrophilic grain disease), neurofibrillary tangle type dementia, diffuse neurofibrillary tangles with calcification (DNTC), and primary age-related tauopathy (PART).

Examples of the test substance include a protein, a peptide, an antibody, a nucleic acid (a gene expression vector, an siRNA, an antisense oligonucleotide, an mRNA), a viral vector (AAV, a lentivirus, an adenovirus, or the like), a non-peptide compound, a synthetic compound, a synthetic low-molecular-weight compound, a natural compound, a cell extract, a plant extract, an animal tissue extract, plasma, an extract derived from a marine organism, a cell culture supernatant, and a microbial fermentation product.

In addition, the test substance can be obtained by using any one of many approaches in combinatorial library methods known in the related art, including (1) a biological library method, (2) a synthetic library method using a deconvolution, (3) a one-bead one-compound library method, and (4) an affinity chromatography selection. The biological library method using affinity chromatography selection is limited to peptide libraries; however, other approaches can be applied to low-molecular-weight compound libraries of peptides, non-peptide oligomers, or compounds (Lam (1997) Anticancer Drug Des. 12: 145-67). Examples of the method of synthesizing molecule libraries can be found in the art (DeWitt et al. (1993) Proc. Natl. Acad. Sci. USA 90: 6909-13; Erb et al. (1994) Proc. Natl. Acad. Sci. USA 91: 11422-6; Zuckermann et al. (1994) J. Med. Chem. 37: 2678-85; Cho et al. (1993) Science 261: 1303-5; Carell et al. (1994) Angew. Chem. Int. Ed. Engl. 33:2059; Carell et. al. (1994) Angew. Chem. Int. Ed. Engl. 33:2061; Gallop et al. (1994) J. Med. Chem. 37: 1233-51). The compound libraries can be prepared as solutions (see Houghten (1992) Bio/Techniques 13: 412-21) or beads (Lam (1991) Nature 354: 82-4), chips (Fodor (1993) Nature 364: 555-6), bacteria (U.S. Pat. No. 5,223,409A), spores (U.S. Pat. Nos. 5,571,698A, 5,403,484A, and 5,223,409A), plasmids (Cull et al. (1992) Proc. Natl. Acad. Sci. USA 89:1865-9), or phages (Scott and Smith (1990) Science 249: 386-90; Devlin (1990) Science 249: 404-6; Cwirla et al. (1990) Proc. Natl. Acad. Sci. USA 87: 6378-82; Felici (1991) J. Mol. Biol. 222: 301-10; US2002/0103360A).

In the present invention, bringing a test substance into contact with the nerve cell may be carried out by adding the test substance to the culture solution of the nerve cell. The contact time is not particularly limited as long as the change of the indicator can be confirmed; however, it is, for example, 1 day or more, 2 days or more, 3 days or more, 4 days or more, 5 days or more, 6 days or more, or 7 days or more. The concentration of the test substance to be added can be appropriately adjusted depending on the kind (in terms of solubility, toxicity, or the like) of the compound.

The culture medium of the nerve cell, which is used in a case where a test substance is brought into contact with the nerve cell, is not particularly limited as long as it is a medium in which the nerve cell is capable of being cultured; however, examples thereof include the above-described medium for nerve differentiation.

The culture temperature at the time of bringing a test substance with the nerve cells is not particularly limited; however, it is about 30° C. to 40° C. and preferably about 37° C. The culture is carried out in an atmosphere of the CO₂-containing air, where the CO₂ concentration is preferably about 2% to 5%.

The present invention will be more specifically described using Examples below; however, the present invention is not limited by Examples.

### Examples

(1) Preparation of Lentivirus Vector

A lentivirus was prepared according to the method described in Mitsuru Ishikawa, et al., Cells 2020, 9, 532. The brief description of the preparation was as follows. Three kinds of plasmids of a packaging construct (pCAG-HIVgp), VSV-G, and a Rev expression construct (pCMV-VSV-G-RSV-Rev), a self-inactivating (SIN) lentivirus vector construct (CSIV-miR-9/9*-124-mRFP1-TRE-EF-B sdT or CSII-TRE-hTau (1N4R)-IRES-Zeo) were transfected into HEK293T cells by using polyethyleneimine (Polysciences, Inc.) to produce a lentivirus. Further, the culture supernatant was concentrated by ultracentrifugation to concentrate the lentivirus. After the concentration, the titer was measured by using lenti-Gostix PLUS (Takara Bio Inc.) and the lentivirus was used in the experiment.

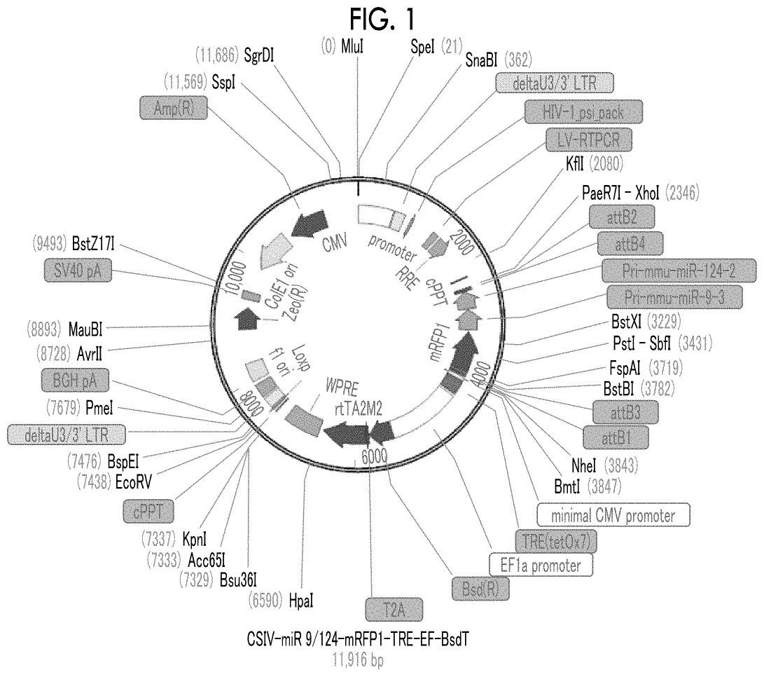

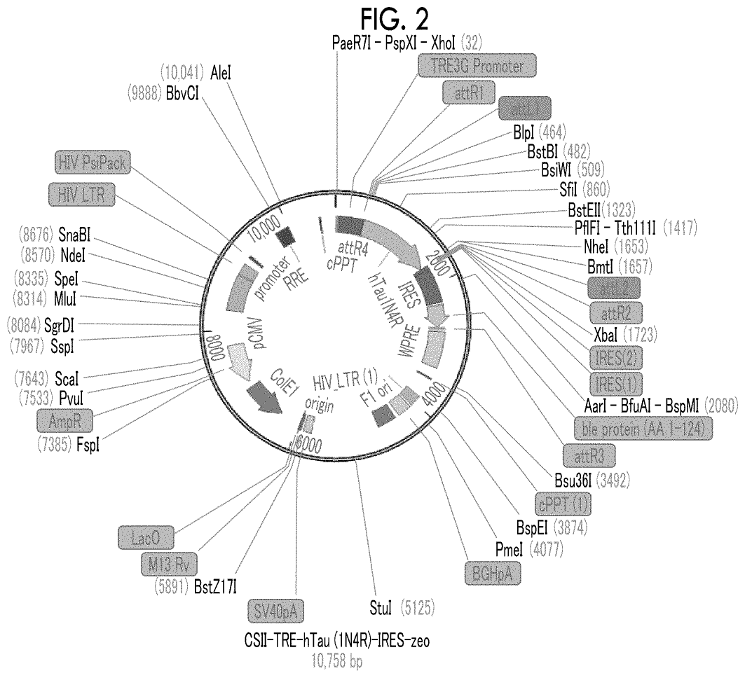

The CSIV-miR-9/9*-124-mRFP1-TRE-EF-BsdT are shown in FIG. 1, and the CSII-TRE-hTau (1N4R)-IRES-Zeo is shown in FIG. 2. For the CSII-TRE-hTau (1N4R)-IRES-Zeo, first, primers including respective attL1 and attL2 as well as respective sequences at both ends of the tau gene sequence were designed, PCR was carried out using the tau gene as a template to create a PCR product having attL1 and attL2 at both ends thereof. Then, in order to create a plasmid by using the Gateway method, reverse PCR was carried out using a pDONR (Thermo Fisher Scientific, Inc.) vector and the above PCR product as a template to create a pDONR vector containing a tau gene. Then, this vector and a CSII vector were subjected to gene recombination using the Gateway method to create CSII-TRE-hTau (1N4R)-IRES-Zeo.

(2) Introduction of TET-on Inducible Ngn2 and miR-9/9*-124 Vectors into iPS Cell

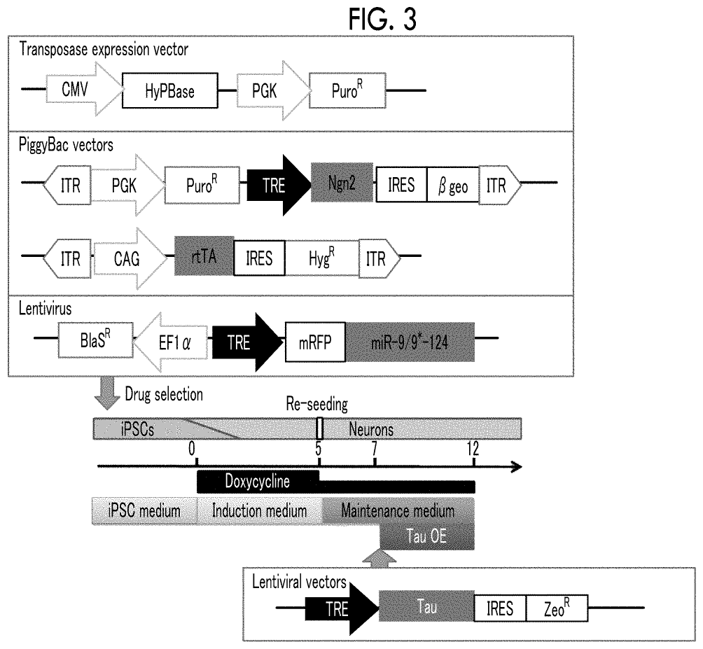

According to the method of Mitsuru Ishikawa, et al., Cells 2020, 9, 532, a transposase vector (pCMV-HyPBase-PGK-Puro) as shown in FIG. 3, a rtTA vector (PB-CAGrtTA3G-IH), and a neurogenin 2 (Ngn2) vector (PB-TET-PH-lox66FRT-NEUROG2) were prepared. These vectors were introduced into an iPS cell cultured with StemFit (registered trade name) by lipofection using GeneJuice (Merck KGaA), and further, a lentivirus (CSIV-miR-9/9*-124-mRFP1-TRE-EF-BsdT) containing the miR-9/9*-124 gene was introduced into the iPS cells. Then, iPS cell lines into which the vectors were stably introduced were obtained by drug selection using puromycin, hygromycin, and blasticidin S.

(3) Preparation of Nerve Cell

iPS cells into which the Ngn2 and miR-9/9*-124 genes had been introduced were detached with TrypLE select and plated on a 6-well plate coated with polyornithine and iMatrix (Nippi, Incorporated). The cells were cultured for 5 days in a neural induction medium containing doxycycline (a medium obtained by adding 2% of a B27 Plus supplement, 1% of a Culture One supplement, 200 μmol/L of dbcAMP, 200 μmol/L of L-ascorbic acid, 10 μmol/L of Y27632, 10 μmol/L of N—[N-(3,5-difluorophenacetyl-L-alanyl)]-(S)-phenylglycine t-butyl ester (DAPT), and 4 μg/ml of DOX to a Neurobasal plus medium), to induce differentiation into nerve cells. The nerve cells were detached with TrypLE Select and used to make a frozen cell stock with Stem Cell Banker.

(4) Lentivirus Vector Infection of iPS Cell-Derived Nerve Cell and Overexpression of Tau

Frozen nerve cells were thawed in a hot water bath and plated on a 96-well plate at the number of cells of about 3×10⁴ cells/well or on a 12-well plate at about 30×10⁴ cells/well, where the plates had been coated with poly-D-lysine (PDL) and iMatrix (Nippi, Incorporated), in a neural replating medium containing DOX (a medium obtained by adding 2% of a B27 Plus supplement, 1% of a Culture One supplement, 200 μmol/L of dbcAMP, 200 μmol/L of L-ascorbic acid, and 2 μg/mL of DOX to a Neurobasal plus:DMEM/F12 HAM=1:1 medium). In addition, 1 μmol/L of Tau Accell siRNA (Dharmacon, Inc., #A-012488-13) described in (7) below or a compound described in (9) below was added thereto. After the culture in a CO₂ incubator at 37° C. for 2 days, the cells were with a lentivirus vector and cultured for about 7 days, and then various evaluations were carried out.

(5) Detection of Overexpressed Tau in hiPSC-Derived Nerve Cell

The nerve cells were infected with the tau-expressing lentivirus vector according to the method of (4) cultured for 5 days, and then the nerve cells were solubilized using a RIPA buffer. SDS-PAGE was carried out using a NuPAGE gel (Thermo Fisher Scientific, Inc.). The proteins on the gel after electrophoresis was transferred to a polyvinylidene fluoride (PVDF) membrane, and after blocking, the membrane was reacted with a tau antibody (manufactured by Agilent Technologies, Inc., diluted to 2,000 times, 4° C., overnight) and an anti-rabbit secondary antibody, 800 nm (purchased from LI-COR Biosciences, diluted to 2,000 times, room temperature, 1 hour) respectively, and the fluorescence was detected with Odyssey CLx (LI-COR Biosciences). The results are shown in FIG. 2. It was confirmed that an amount of tau equal to or larger than that of endogenous tau is induced by overexpression (OE).

(6) Degeneration of Neurite Due to Overexpression of Tau in hiPSC-Derived Nerve Cell

The nerve cells were infected with the tau-expressing lentivirus vector according to the method of (4), and then the cells were fixed with 4% paraformaldehyde 6 days later. After blocking, a primary antibody (diluted to 1,000 times, 4° C., overnight) and a secondary antibody (diluted to 2,000 times, room temperature, 1 hour) were reacted respectively, and the fluorescence was detected with a fluorescence microscope. The results are shown in FIG. 3. The overexpression of 1N4R tau increased the signals of 4R tau and phosphorylated tau (AT-8, PHF-1). Furthermore, fragmentation of 0111 tubulin and reduction of MAP2 neurites were observed by the overexpression of tau, and thus it was confirmed that degeneration of neurites has occurred.

The antibodies used are shown below.

Rabbit 4R tau antibody (Cosmo Bio Co., Ltd.)

Mouse AT-8 antibody (Thermo Fisher Scientific, Inc.)

Mouse PHF antibody (provided by Dr. Peter Davies)

Rabbit βIII-tubulin antibody (Abcam plc)

Mouse MAP2 antibody (Sigma-Aldrich Co., LLC)

Anti-mouse IgG Alexa Fluor 555 (Thermo Fisher Scientific, Inc.)

Anti-rabbit IgG Alexa Fluor 647 (Thermo Fisher Scientific, Inc.)

(7) Nerve Cell Death Due to Overexpression of Tau

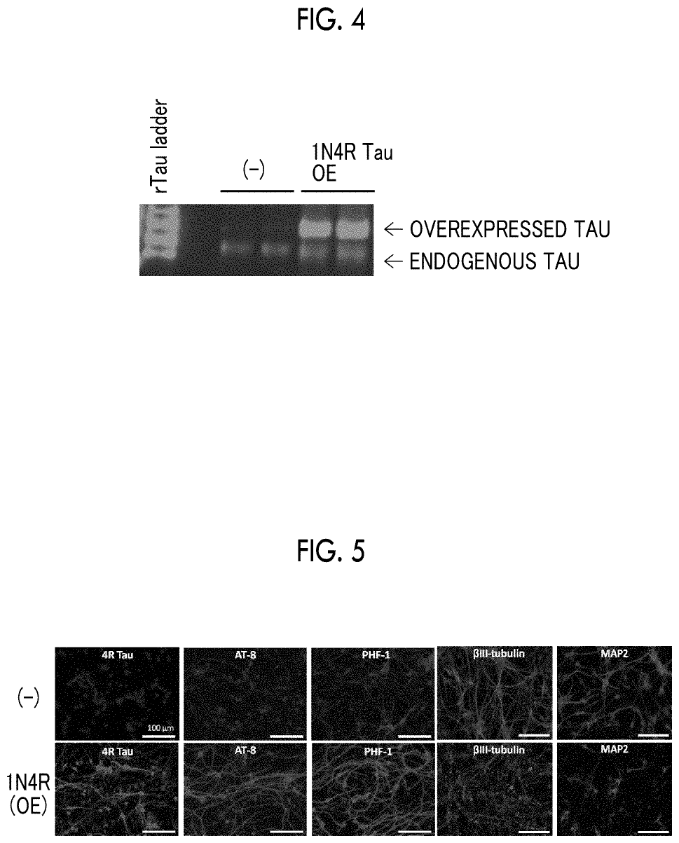

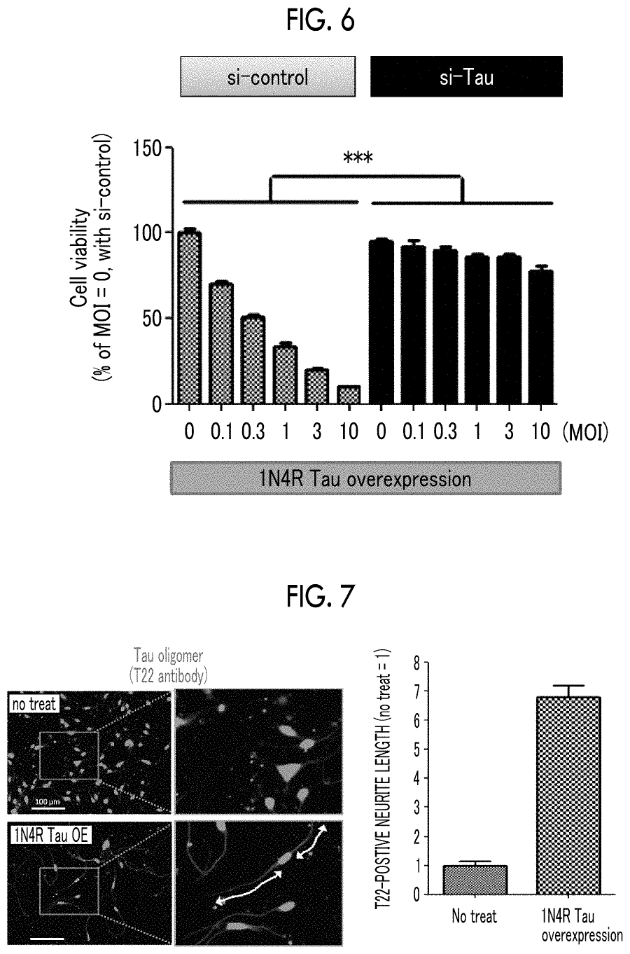

The decrease in cell survival rate due to overexpression of tau according to the method (4) was confirmed by the CellTiter Glo assay (Promega Corporation). The results are shown in FIG. 4. It was confirmed that about 20% to 90% of nerve cell death occurred due to overexpression of tau depending on the amount of lentivirus. Further, the treatment with Tau Accell siRNA (Dharmacon, Inc., #D-001910-03) suppressed nerve cell death, indicating that nerve cell death occurs in a tau expression-dependent manner.

(8) Tau Aggregation Due to Overexpression of Tau

The nerve cells after the overexpression of tau according to the method (4) were subjected to fluorescent staining using a tau oligomer antibody (T22) (Merck Millipore). The results are shown in FIG. 7. Tau oligomer-positive neurites were confirmed in the staining image. This indicates that tau aggregation occurs in nerve cells. Furthermore, when the T22 antibody-positive neurite length was quantified using Image J, an increase in T22-positive neurite length was confirmed in the tau overexpressing group as compared with the non-treated group.

(9) Neuroprotective Action by Tau Aggregation Inhibitor and Microtubule Stabilizer

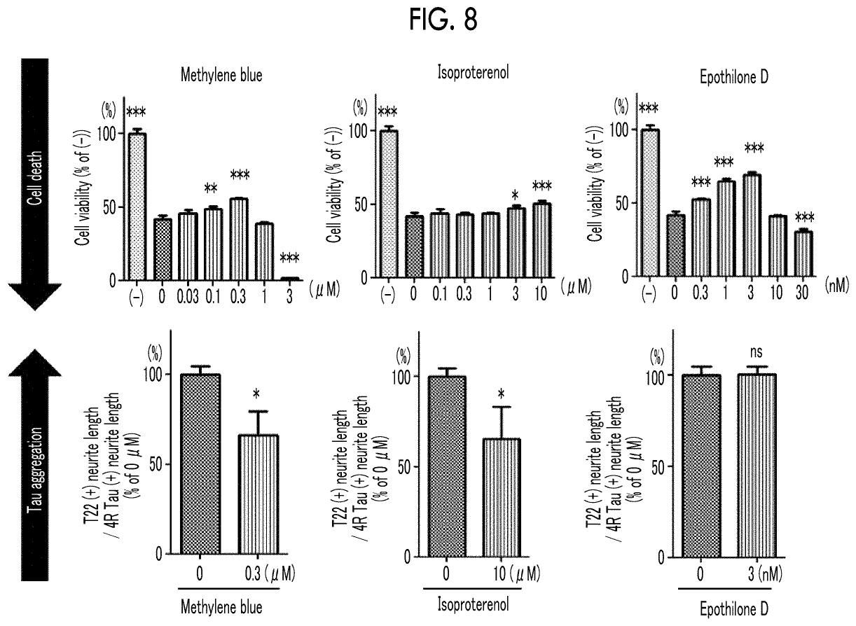

According to the method (4), the drug efficacy of methylene blue and isoproterenol, which had been reported to have an inhibitory action on tau aggregation in nerve cells after the overexpression of tau, and a microtubule stabilizer epothilone D was evaluated. The cytoprotective action of the compound was measured by a CellTiter Glo assay. The effect on tau aggregation was quantified by standardizing the Tau oligomer antibody-positive protrusion length in the neurite obtained by fluorescent staining with the 4R tau antibody-positive protrusion length. The results are shown in FIG. 8. A partial cytoprotective action was confirmed in methylene blue, isoproterenol, and epothilone D. Further, tau polymerization was evaluated at the compound concentration at which a cytoprotective action was exhibited. A partial inhibitory action on tau aggregation was confirmed in methylene blue and isoproterenol, which had been reported to have an inhibitory action on tau polymerization. On the other hand, tau polymerization was not changed by the microtubule stabilizer epothilone D, and a cytoprotective action was exhibited in a tau polymerization-independent manner.

What is claimed is:

1. A nerve cell comprising an introduced exogenous wild-type tau gene.

2. The nerve cell according to claim 1,
wherein the nerve cell is derived from a human pluripotent stem cell.

3. The nerve cell according to claim 2,
wherein the human pluripotent stem cell is a human pluripotent stem cell derived from a healthy subject.

4. The nerve cell according to claim 2,
wherein the exogenous wild-type tau gene is expressed after differentiation of the human pluripotent stem cells into the nerve cell.

5. The nerve cell according to claim 3,
wherein the exogenous wild-type tau gene is expressed after differentiation of the human pluripotent stem cells into the nerve cell.

6. The nerve cell according to claim 1,
wherein an expression of the exogenous wild-type tau gene is controlled in a condition-specific manner.

7. The nerve cell according to claim 2,
wherein an expression of the exogenous wild-type tau gene is controlled in a condition-specific manner.

8. The nerve cell according to claim 3,
wherein an expression of the exogenous wild-type tau gene is controlled in a condition-specific manner.

9. The nerve cell according to claim 1,
wherein tau aggregates in a case where the exogenous wild-type tau gene is overexpressed.

10. The nerve cell according to claim 2,
wherein tau aggregates in a case where the exogenous wild-type tau gene is overexpressed.

11. The nerve cell according to claim 3,
wherein tau aggregates in a case where the exogenous wild-type tau gene is overexpressed.

12. The nerve cell according to claim 1,
wherein aggregation of tau occurs due solely to a gene expression.

13. The nerve cell according to claim 2,
wherein aggregation of tau occurs due solely to a gene expression.

14. The nerve cell according to claim 3,
wherein aggregation of tau occurs due solely to a gene expression.

15. A screening kit for a drug candidate substance having an action on a change due to overexpression of tau, the screening kit comprising the nerve cell according to claim 1.

16. A screening method for a drug candidate substance having an action of suppressing tau aggregation or cell death, the screening method comprising using the nerve cell according to claim 1.

17. A drug candidate substance obtained by the screening method according to claim 16.

18. A human pluripotent stem cell comprising an introduced exogenous wild-type tau gene.

19. A method of producing a nerve cell, comprising:
introducing an exogenous wild-type tau gene into a human pluripotent stem cell; and
differentiating the human pluripotent stem cell into a nerve cell.

20. The method according to claim 19,
wherein steps of an expression of the exogenous wild-type tau gene and a gene expression for nerve cell differentiation are provided by a TET system.
